(19) United States
(12) Patent Application Publication
(10) Pub. No.: US 2020/0300873 A1
(43) Pub. Date: Sep. 24, 2020

(54) KITS FOR DETECTION OF HEPCIDIN

(71) Applicant: Intrinsic LifeSciences LLC, La Jolla, CA (US)

(72) Inventors: Mark Westerman, La Jolla, CA (US); Patrick Gutschow, Poway, CA (US); Vaughn Ostland, San Diego, CA (US)

(21) Appl. No.: 15/774,549

(22) PCT Filed: Nov. 21, 2016
(86) PCT No.: PCT/US16/63009

§ 371 Date: May 8, 2018

Related U.S. Application Data
(60) Provisional application No. 62/260,106, filed on Nov. 25, 2015.
(60) Provisional application No. 62/263,245, filed on Dec. 4, 2015.

(51) Int. Cl. G01N 33/74; C07K 16/26; C07K 14/575; G01N 33/68; G01N 33/53

(52) U.S. Cl. CPC G01N 33/74; C07K 16/26; C07K 14/575; G01N 2800/04; G01N 33/5304; C07K 2317/56; G01N 2333/575; G01N 33/6872

(57) ABSTRACT

The present invention is generally related to kits for measuring a level of hepcidin in a biological sample in an in vitro assay compared to a standard control. The kits may further include instructions for use, a label for use, or both. The kits may be used to identify an individual having a disease or disorder associated with an elevated level of hepcidin, a reduced level of hepcidin, an elevated level of iron, a reduced level of iron, or a combination thereof.

### CROSS-REFERENCE

This application claims the benefit of U.S. Provisional Application No. 62/260,106, filed on Nov. 25, 2015, and U.S. Provisional Application No. 62/263,245, filed on Dec. 4, 2015, which are incorporated by reference in their entirety.

### SEQUENCE LISTING

The instant application contains a Sequence Listing which has been submitted electronically in ASCII format and is hereby incorporated by reference in its entirety. Said ASCII copy, created on Nov. 18, 2016, is named 44546-710_601_SL.txt and is 4,024 bytes in size.

### BACKGROUND OF THE INVENTION

Iron is an essential trace element required for growth and development of living organisms. In mammals, iron content is regulated by controlling iron absorption, iron recycling, and release of iron from the cells in which it is stored. Iron is predominantly absorbed in the duodenum and upper jejunum by enterocytes. Iron is recycled from degraded red cells by reticuloendothelial macrophages in bone marrow, hepatic Kupffer cells and spleen. Iron release is controlled by ferroportin, a major iron export protein located on the cell surface of enterocytes, macrophages and hepatocytes, the main cells capable of releasing iron into plasma. Hepcidin binds to ferroportin and decreases its functional activity by causing it to be internalized from the cell surface and degraded.

### SUMMARY OF THE INVENTION

The Intrinsic Hepcidin IDx™ ELISA kit is an ELISA test for the quantitative measurement of hepcidin in human serum and plasma.

Provided herein is a kit comprising a) a 96-microwell plate pre-coated with an anti-hepcidin antibody; b) a hepcidin-25 standard; c) a first hepcidin-25 control; d) a second hepcidin-25 control; e) a biotinylated hepcidin-25 tracer; f) a streptavidin horseradish peroxidase conjugate; g) a TMB substrate; h) a stop solution; i) a wash buffer; and j) a sample diluent.

A microwell plate may be, for example, a polypropylene or a polystyrene microwell plate. In some instances, the microwell strip plate is a polystyrene microwell plate. The microwell plate may be a strip plate or a solid plate.

In some instances, the kit further comprises two adhesive covers for the microwell strip plate.

In some instances, the antibody of a) is a monoclonal antibody having a variable heavy chain set forth as SEQ ID NO: 5 and a variable light chain set forth as SEQ ID NO: 7.

The kit may comprise up to 8 vials of a hepcidin-25 standard. In some instances, the kit comprises 8 vials of a hepcidin-25 standard. For example, each vial can comprise 0.5 mL of hepcidin-25 standard.

In some instances, the kit comprises 1 vial of a first hepcidin-25 control. For example, each vial of the first hepcidin-25 control can comprise 0.5 mL of reagent.

In some instances, the kit comprises 1 vial of a second hepcidin-25 control. For example, each vial of the second hepcidin-25 control can comprise 0.5 mL of reagent.

The kit may comprise 1 bottle of biotinylated hepcidin-25 tracer. For example, each vial can comprise 12 mL of biotinylated hepcidin-25 tracer. In some instances, the biotinylated hepcidin-25 tracer consists of: (i) a hepcidin peptide that is oxidatively folded; (ii) a hydrophilic spacer consisting of two (2-(2-Amino-Ethoxy) Ethoxy) Acetic Acid (AEEAc) residues, wherein the peptide of (i) is covalently linked to the hydrophilic spacer at the amino terminus of the peptide; and (iii) biotin covalently linked to the hydrophilic spacer of (ii). In some instances, the hepcidin peptide has an amino acid sequence set forth as SEQ ID NO: 1.

In some instances, the kit comprises 1 bottle of Streptavidin-HRP Conjugate. For example, the bottle can comprise 12 mL of Streptavidin-HRP Conjugate.

In some instances, the kit comprises 1 bottle of TMB Substrate. For example, the bottle can comprise 12 mL of TMB Substrate.

In some instances, the kit comprises 1 bottle of Stop Solution. For example, the bottle can comprise 12 mL of Stop Solution.

In some instances, the kit comprises 1 bottle of Wash Buffer. For example, the bottle can comprise 25 mL of Wash Buffer. In certain instances, the kit can comprise a 20× solution of the Wash Buffer where the wash solution is diluted prior to use in the kit.

In some instances, the kit comprises 1 bottle of Sample Diluent. For example, the bottle can comprise 3 mL of Sample Diluent.

In some aspects, the kit further comprises instructions for use.

The instructions can include, for example, identification of a biological sample for use in the kit.

A kit can further comprise one or more collection means for collection of the biological sample. For example, the one or more collection means can comprise a syringe, a needle, a cup, a swab or a combination thereof. Other collection means are known in the art and are contemplated herein.

In some aspects, a biological sample comprises a blood sample, a tissue sample or a urine sample. The instructions may also include instructions for treating a biological sample prior to use in the kit. Blood can be treating using conventional methods. For example, heparin or EDTA may be added to a blood sample. Serum may also be obtained from a blood sample and used in the described assay kits. Blood may be centrifuged to remove cellular components.

In some aspects, the kit further comprises a label.

The kit can be used for detecting one or more diseases or disorders associated with an elevated level of hepcidin, a reduced level of hepcidin, an elevated level of iron, a reduced level of iron, or a combination thereof. Non-limiting examples of diseases or disorders associated with elevated level of hepcidin, a reduced level of hepcidin, an elevated level of iron, a reduced level of iron, or a combination thereof, include, but are not limited to, African iron overload, alpha thalassemia, Alzheimer's disease, anemia, anemia of cancer, anemia of chronic disease, anemia of inflammation, arteriosclerosis or atherosclerosis, ataxias, ataxias related to iron, atransferrinemia, cancer, ceruloplasmin deficiency, chemotherapy-induced anemia, chronic renal disease, including end stage renal disease or chronic renal/kidney failure, acute kidney injury (AKI), cirrhosis of liver, classic hemochromatosis, collagen-induced arthritis (CIA), congenital dyserythropoietic anemia, congestive heart failure, Crohn's disease, Celiac disease, inflammatory bowel disease (IBD), diabetes, a disorder of iron biodistribution, a disorder of iron homeostasis, a disorder of iron metabolism, ferroportin disease, ferroportin mutation hemochromatosis, folate deficiency, Friedrich's ataxia, funicular myelosis, gracile syndrome, a bacterial infection, Hallervordan Spatz disease, hemochromatosis, hemochromatosis resulting from mutations in transferrin receptor 2, hemoglobinopathies, hepatitis, hepatocellular carcinoma, hereditary hemochromatosis, a viral infection, Huntington's disease, hyperferritinemia, hypochromic microcytic anemia, hypoferremia, insulin resistance, iron deficiency anemia, an iron deficiency disorder, an iron overload disorder, an iron-deficiency condition with hepcidin excess, juvenile hemochromatosis (HFE2), multiple sclerosis, mutation in transferrin receptor 2, HFE, hemojuvelin, ferroportin, TMPRSS6 (IRIDA), or other genes of iron metabolism, neonatal hemochromatosis, neurodegenerative diseases related to iron, osteopenia, osteoporosis pancreatitis, Pantothenate kinase-associated neurodegeneration, Parkinson's disease, pellagra, pica, porphyria, porphyria cutanea tarda, pseudoencephalitis, pulmonary hemosiderosis, a red blood cell disorder, rheumatoid arthritis, sepsis, sideroblastic anemia, systemic lupus erythematosus, thalassemia, thalassemia intermedia, transfusional iron overload, a tumor, vasculitis, vitamin B6 deficiency, vitamin B12 deficiency, Wilson's disease or a combination thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DISCLOSURE OF THE DRAWINGS

The novel features of the invention are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates the relationship between the hepcidin concentration of human blood samples (n=8) collected and prepared as lithium heparin (Li-heparin) and measured using the Intrinsic LifeSciences (ILS) Hepcidin IDx™ ELISA kit and the DRG® Hepcidin-25 Bioactive ELISA kit.

FIG. 2 illustrates the relationship between the hepcidin concentration of the DRG® Hepcidin-25 Bioactive ELISA kit standards and the hepcidin concentration of the standards in the ILS Hepcidin IDx™ ELISA kit. The ILS Hepcidin IDx™ ELISA kit determined that the DRG® hepcidin standards included in the DRG® Hepcidin-25 Bioactive ELISA kit contain approximately 10-fold less hepcidin than is described in the DRG® manufacturer's instructions.

FIG. 3 illustrates the relationship between the hepcidin concentration of the ILS Hepcidin IDx™ ELISA kit standards and the hepcidin concentration of the standards included in the DRG® Hepcidin-25 Bioactive ELISA kit. These results confirm the data of FIG. 2 because the DRG® Hepcidin-25 Bioactive ELISA kit measures approximately 10-fold more hepcidin in the ILS Hepcidin IDx™ ELISA kit standards than in the standards of the DRG® Hepcidin-25 Bioactive ELISA kit.

FIGS. 4A-4B: FIG. 4A illustrates the relationship between the hepcidin concentration in human blood samples (n=8) collected and prepared as EDTA plasma (dashed line) or lithium heparin (Li-heparin, solid line) or serum and measured with the ILS Hepcidin IDx™ ELISA kit. FIG. 4B illustrates the relationship between the hepcidin concentration in human blood samples (n=8) collected and prepared as EDTA plasma (dashed line) or lithium heparin (Li-heparin, solid line) or serum and measured with the DRG® Hepcidin-25 Bioactive ELISA kit.

### DETAILED DESCRIPTION OF THE INVENTION

Precision and accuracy are among the most important functional characteristics of any assay. Accuracy is difficult to assess if the assay suffers from poor reproducibility and/or if the standards are not the concentration disclosed in the kit. The ability to reproduce a sample measurement from one well to another in a single assay and a sample measurement from one assay to the next is the first step to understanding the viability of the assay. Immunoassays on the market generally have an intra- and inter-assay coefficient of variation (CV) below 20%. The assays and kits described in the present application are significantly improved compared to this percentage.

Hepcidin is a 25 amino acid hormone and is the master regulator for iron homeostasis (metabolism) in humans. Hepcidin regulates dietary iron absorption from the duodenum, controls the recycling of senescent erythrocyte iron by macrophages, and manages iron transport from hepatocytes into plasma for production of blood. Hepcidin is positively regulated by plasma iron and IL-6 (inflammation, infection), and is suppressed by erythropoiesis via erythroferrone. Abnormally low serum hepcidin is associated with iron deficiency anemia (IDA) and hereditary hemochromatosis, and high serum hepcidin can lead to iron sequestration and to anemia of inflammation (anemia of chronic disease) observed in chronic kidney disease (CKD), rheumatoid arthritis (RA), cancers, and iron refractory iron deficiency anemia (IRIDA). Recently hepcidin was shown to be useful in predicting response to oral iron therapy based on the hepcidin level and, for this indication, it is superior to both ferritin and Percent (%) transferrin saturation (% Tsat).

Hepcidin is involved in regulating iron homeostasis. Hepcidin binds to ferroportin and decreases its functional activity by causing it to be internalized from the cell surface and degraded.

High levels of human hepcidin may result in reduced iron levels, and vice versa. Mutations in the hepcidin gene which result in lack of hepcidin activity are associated with juvenile hemochromatosis, a severe iron overload disease. Studies in mice have demonstrated a role of hepcidin in control of normal iron homeostasis.

Hepcidin may also be involved in iron sequestration during inflammation. Hepcidin gene expression has been observed to be robustly up-regulated after inflammatory stimuli, such as infections, which induce the acute phase response of the innate immune systems of vertebrates. Hepcidin gene expression may be up-regulated by lipopolysaccharide (LPS), turpentine, Freund's complete adjuvant, incomplete adjuvant, adenoviral infections and the inflammatory cytokine interleukin-6 (IL-6). A strong correlation between hepcidin expression and anemia of inflammation was also found in patients with chronic inflammatory diseases, including bacterial, fungal, and viral infections.

Human hepcidin is a 25 amino acid peptide with anti-microbial and iron-regulating activity. It has also been referred to as LEAP-1 (liver-expressed antimicrobial peptide). A hepcidin cDNA encoding an 83 amino acid pre-propeptide in mice and an 84 amino acid pre-propeptide in rat and human were subsequently identified in a search for liver specific genes that were regulated by iron. The 24 residue N-terminal signal peptide is first cleaved to produce pro-hepcidin, which is then further processed to produce mature hepcidin, found in both blood and urine. In human urine, the predominant form contains 25 amino acids, although shorter 22 and 20 amino acid peptides are also present at undetectable or very low concentrations in certain diseases.

A kit described herein can be used to detect levels of mature human hepcidin in a biological sample.

“About”, as used herein, generally means a range spanning an indicated value up to ±0.5%, ±1%, ±1.5%, ±2%, ±2.5%, ±3%, ±3.5%, ±4%, ±5.5%, ±6%, ±6.5%, ±7%, ±7.5%, ±8%, ±8.5%, ±9%, ±9.5%, ±10%, ±11%, ±12%, ±13%, ±14%, ±15%, ±16%, ±17%, ±18%, ±19%, or ±20% of the indicated value, or any value therebetween.

### Antibodies

In one aspect, provided herein is an antibody that specifically binds to hepcidin or a mature human hepcidin peptide, comprising a heavy chain variable region and a light chain variable region. In some embodiments, the antibody binds to the N-terminus of a mature human hepcidin 25 amino acid peptide.

In one instance, an antibody for use in a kit provided herein comprises a heavy chain variable region comprising an amino acid sequence of SEQ ID NO: 5 and a light chain variable region comprising an amino acid sequence of SEQ ID NO: 7.

In one aspect, an antibody provided herein comprises IgG1 or an IgG4 variable heavy chain and variable light chain.

An antibody described herein may have a dissociation constant (K_{d}) of from about 1 to about 500 pM, from about 1 to about 10 pM, from about 10 to about 20 pM, from about 1 to about 29 pM, from about 30 to about 40 pM, from about 10 to about 100 pM, or from about 20 to about 500 pM.

An antibody described herein may have a dissociation constant (K_{d}) of less than about 500 pM, less than about 450 pM, less than about 400 pM, less than about 350 pM, less than about 300 pM, less than about 250 pM, less than about 200 pM, less than about 150 pM, less than about 100 pM, less than about 75 pM, less than about 50 pM, less than about 30 pM, less than about 25 pM, less than about 20 pM, less than about 18 pM, less than about 15 pM, less than about 10 pM, less than about 7.5 pM, less than about 5 pM, less than about 2.5 pM, or less than about 1 pM.

An antibody described herein may have an affinity for a hepcidin peptide of from about 10⁻⁹ to about 10⁻¹⁴, from about 10⁻¹⁰ to about 10⁻¹⁴, from about 10⁻¹¹ to about 10⁻¹⁴, from about 10⁻¹² to about 10⁻¹⁴, from about 10⁻¹³ to about 10⁻¹⁴, from about 10⁻¹⁰ to about 10⁻¹¹, from about 10⁻¹¹ to about 10⁻¹², from about 10⁻¹² to about 10⁻¹³, or 10⁻¹³ to about 10⁻¹⁴

The Intrinsic Hepcidin IDx™ ELISA kit is a competitive binding assay based on a monoclonal antibody (mAb) that binds with high affinity to the N-terminus of hepcidin-25 (approximately 5.16×10⁻¹¹), which is required for bioactivity and binding to ferroportin. This antibody also binds low abundance, N-terminus isomers of hepcidin-25 with lower affinities. Intrinsic LifeSciences developed the described ELISA test for hepcidin that is capable of measuring clinical levels of hepcidin in key clinical iron disorders.

Provided herein is a composition, comprising an antibody described herein and an acceptable buffer.

The terms “hypervariable region” and “CDR” when used herein, refer to the amino acid residues of an antibody which are responsible for antigen-binding. The CDRs comprise amino acid residues from three sequence regions which bind in a complementary manner to an antigen and are known as CDR1, CDR2, and CDR3 for each of the V_{H} and V_{L} chains. In the light chain variable domain, the CDRs typically correspond to approximately residues 24-34 (CDRL1), 50-56 (CDRL2) and 89-97 (CDRL3), and in the heavy chain variable domain the CDRs typically correspond to approximately residues 31-35 (CDRH1), 50-65 (CDRH2) and 95-102 (CDRH3) according to Kabat et al., Sequences of Proteins of Immunological Interest, 5th Ed. Public Health Service, National Institutes of Health, Bethesda, Md. (1991)). It is understood that the CDRs of different antibodies may contain insertions, thus the amino acid numbering may differ. The Kabat numbering system accounts for such insertions with a numbering scheme that utilizes letters attached to specific residues (e.g., 27A, 27B, 27C, 27D, 27E, and 27F of CDRL1 in the light chain) to reflect any insertions in the numberings between different antibodies. Alternatively, in the light chain variable domain, the CDRs typically correspond to approximately residues 26-32 (CDRL1), 50-52 (CDRL2) and 91-96 (CDRL3), and in the heavy chain variable domain, the CDRs typically correspond to approximately residues 26-32 (CDRH1), 53-55 (CDRH2) and 96-101 (CDRH3) according to Chothia and Lesk, J. Mol. Biol., 196: 901-917 (1987)).

As used herein, “framework region” or “FR” refers to framework amino acid residues that form a part of the antigen binding pocket or groove. In some embodiments, the framework residues form a loop that is a part of the antigen binding pocket or groove and the amino acids residues in the loop may or may not contact the antigen. Framework regions generally comprise the regions between the CDRs. In the light chain variable domain, the FRs typically correspond to approximately residues 0-23 (FRL1), 35-49 (FRL2), 57-88 (FRL3), and 98-109 and in the heavy chain variable domain the FRs typically correspond to approximately residues 0-30 (FRH1), 36-49 (FRH2), 66-94 (FRH3), and 103-133 according to Kabat et al., Sequences of Proteins of Immunological Interest, 5th Ed. Public Health Service, National Institutes of Health, Bethesda, Md. (1991)). As discussed above with the Kabat numbering for the light chain, the heavy chain too accounts for insertions in a similar manner (e.g., 35A, 35B of CDRH1 in the heavy chain). Alternatively, in the light chain variable domain, the FRs typically correspond to approximately residues 0-25 (FRL1), 33-49 (FRL2) 53-90 (FRL3), and 97-109 (FRL4), and in the heavy chain variable domain, the FRs typically correspond to approximately residues 0-25 (FRH1), 33-52 (FRH2), 56-95 (FRH3), and 102-113 (FRH4) according to Chothia and Lesk, J. Mol. Biol., 196: 901-917 (1987)).

The loop amino acids of a FR can be assessed and determined by inspection of the three-dimensional structure of an antibody heavy chain and/or antibody light chain. The three-dimensional structure can be analyzed for solvent accessible amino acid positions as such positions are likely to form a loop and/or provide antigen contact in an antibody variable domain. Some of the solvent accessible positions can tolerate amino acid sequence diversity and others (e.g., structural positions) are, generally, less diversified. The three dimensional structure of the antibody variable domain can be derived from a crystal structure or protein modeling.

Constant domains (Fc) of antibodies are not involved directly in binding an antibody to an antigen but, rather, exhibit various effector functions, such as participation of the antibody in antibody-dependent cellular toxicity via interactions with, for example, Fc receptors (FcR). Fc domains can also increase bioavailability of an antibody in circulation following administration to a subject.

The term “specific” refers to a situation in which an antibody will not show any significant binding to molecules other than the antigen containing the epitope recognized by the antibody. The term is also applicable where for example, an antigen binding domain is specific for a particular epitope which is carried by a number of antigens, in which case the antibody or antigen-binding fragment thereof carrying the antigen binding domain will be able to bind to the various antigens carrying the epitope. The terms “preferentially binds” or “specifically binds” mean that the antibodies or fragments thereof bind to an epitope with greater affinity than it binds unrelated amino acid sequences, and, if cross-reactive to other polypeptides containing the epitope, are not toxic at the levels at which they are formulated for administration to human use. In one aspect, such affinity is at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater than the affinity of the antibody or fragment thereof for unrelated amino acid sequences. The terms “immunoreactive,” “binds,” “preferentially binds” and “specifically binds” are used interchangeably herein. The term “binding” refers to a direct association between two molecules, due to, for example, covalent, electrostatic, hydrophobic, and ionic and/or hydrogen-bond interactions under physiological conditions, and includes interactions such as salt bridges and water bridges, as well as any other conventional means of binding.

“Isolated” (used interchangeably with “substantially pure” or “purified”) when applied to antibodies means an antibody that is synthesized chemically or expressed in a host cell and purified away from associated and contaminating proteins. The term generally means a polypeptide that has been separated from other proteins and nucleic acids with which it naturally occurs and/or substances which are used to purify it. In some instances, an antibody provided in a kit described herein is about 95%, about 96%, about 97%, about 98%, about 99% or above pure.

### Tracers

Provided herein is a an immunoassay tracer reagent consisting of: (i) a hepcidin peptide that is oxidatively folded; (ii) a hydrophilic spacer consisting of two (2-(2-Amino-Ethoxy) Ethoxy) Acetic Acid (AEEAc) residues, wherein the peptide of (i) is covalently linked to the hydrophilic spacer at the amino terminus (N-terminus) of the peptide; and (iii) biotin covalently linked to the hydrophilic spacer of (ii). In some instances, the hepcidin peptide is oxidatively folded before it is covalently linked to the hydrophilic spacer of (ii). In some instances, the hepcidin peptide that is oxidatively folded has an amino acid sequence of DTHFPICIFCCGCCHRSKCGMCCKT (SEQ ID NO: 1). The hepcidin peptide that is oxidatively folded may have the amino acid sequence of human hepcidin 25.

A hepcidin peptide can be oxidatively folded by a process comprising: (a) solubilizing the hepcidin in an acetic acid solution to produce a first solution; (b) diluting the first solution with an aqueous buffer solution containing a chaotropic reagent, an organic alcohol, and an oxidizing reagent to produce a second solution; and (c) adjusting the pH of the second solution to a level between approximately 5 and 7. The organic alcohol can be approximately 10% isopropyl alcohol; (ii) the pH can be adjusted by the addition of ammonium hydroxide; or (iii) the oxidation may occur at room temperature.

### Kits

Provided herein is a kit comprising a composition described herein where the composition comprises an antibody, an acceptable carrier and excipient, and a label describing steps for use of the kit. In some instances, the kit may also contain instructions for obtaining and processing a sample prior to use in the kit. In one aspect, provided herein is a kit for diagnosing a disorder associated with elevated hepcidin levels or a disorder of iron homeostasis, comprising, a 96-well microwell place coated with an antibody as described herein and blocked with a blocking agent, a biotinylated tracer molecule, streptavidin horseradish peroxidase, a substrate solution, a stop solution, a wash buffer, a sample diluent and a standard (i.e., a positive control). Diluent without standard may be used as a negative control.

Provided herein is a container means comprising a composition described herein. The container means may be any suitable container which may house a liquid or lyophilized composition including, but not limited to, a vial, syringe, bottle, an in intravenous (IV) bag or ampoule. Container means may be made of any appropriate material for storing the kit contents. A container means may be able to hold any volume of liquid suitable for use in a method described herein including, but not limited to, from about 0.1 ml to about 50 ml or more; for example, about 0.5 ml, about 1 ml, about 2 ml, about 5 ml, about 10 ml, about 15 ml, about 20 ml, about 25 ml, about 30 ml, about 35 ml, about 40 ml, about 45 ml or more, or any integer therebetween. In some instances, a container means holds a solid composition or a liquid composition. Where a container means holds a solid composition, the container means may be able to hold any amount of composition for use in a method described herein including, but not limited to, from about 0.01 mg to about 50 g or more; for example, about 0.5 mg, about 1 mg, about 2 mg, about 5 mg, about 10 mg, about 15 mg or more, or any integer therebetween.

The kits may include a means for containing a reagent in close confinement for commercial sale. Such containers may include injection and/or blow-molded plastic containers into which the desired vials are retained. Kits can also include printed material for use of the materials in the kit.

Formulations/preparations included in a package or a kit may additionally include a buffering agent, a preservative, a stabilizing agent or a combination thereof. Each component of the kit can be enclosed within an individual container and all of the various containers can be within a single package. Invention kits can be designed for cold storage or room temperature storage.

Additionally, the reagent formulations/preparations can contain stabilizers to increase the shelf-life of the kits and include, for example, bovine serum albumin (BSA). Where the compositions are lyophilized, the kit may contain further preparations of solutions to reconstitute the lyophilized preparations. Acceptable reconstitution solutions are well known in the art and include, for example, pharmaceutically acceptable phosphate buffered saline (PBS).

Packages and kits can further include one or more components for an assay, such as, for example, an ELISA assay. For example, a kit may include a microwell plate such as a 96-well microwell plate. In some instances, a microwell plate of a kit provided herein is coated with an anti-hepcidin antibody and blocked with a blocking agent prior to packaging. Such plates can be sealed in a sterile plastic wrapper and vacuum sealed. Plates are prepared in sterile conditions to avoid contamination.

Packages and kits can further include one or more components for collection of a sample (e.g., a syringe, a needle, a cup, a vial, a bottle, a swab, etc.).

Packages and kits can further include a label specifying, for example, a product description.

The term “packaging material” refers to a physical structure housing the components of the kit. The packaging material can maintain the components sterilely and can be made of material commonly used for such purposes (e.g., paper, corrugated fiber, glass, plastic, foil, ampules, etc.). The label or packaging insert can include appropriate written instructions. Kits, therefore, can additionally include labels or instructions for using the kit components in any method of the invention. A kit can include a compound in a pack, or dispenser together with instructions for administering the compound in a method described herein.

Instructions can include instructions for practicing any of the methods described herein including diagnostic methods.

The instructions may be on “printed matter,” e.g., on paper or cardboard within or affixed to the kit, or on a label affixed to the kit or packaging material, or attached to a vial or tube containing a component of the kit. Instructions may additionally be included on a computer readable medium, such as a flash/cloud drive, disk (floppy diskette or hard disk), optical CD such as CD- or DVD-ROM/RAM, magnetic tape, electrical storage media such as RAM and ROM, IC tip and hybrids of these such as magnetic/optical storage media.

### Diagnostic Methods

In one embodiment, the antibody or antigen-binding fragment further comprises a detectable moiety. Detection can occur in vitro. In vitro assays for the detection and/or determination (quantification, qualification, etc.) of hepcidin with an antibody described herein includes for example, a competitive Enzyme Linked Immunosorbant Assay (ELISA). In vitro detection, diagnosis or monitoring of hepcidin can occur by obtaining a sample (e.g., a blood sample, a tissue sample or a urine sample) from a subject and testing the sample in, for example, a competitive ELISA assay as described, for example, in the Examples below.

Provided herein is a method of diagnosing a hepcidin-related disorder, comprising: (a) contacting a biological sample from a subject suspected of having said disorder with an antibody, or antigen-binding fragment thereof, described herein under conditions that allow binding of the antibody or antigen-binding fragment thereof, to hepcidin; and (b) detecting and/or quantitating the hepcidin bound to the antibody, or antigen-binding fragment thereof, wherein the amount of hepcidin in the sample, as quantitated in (b), above a threshold level indicates the presence of hepcidin-related disorder and below the threshold level indicates the absence of hepcidin-related disorder. In some instances, the biological sample is treated/processed prior to use in the described kit assay. For example, EDTA or heparin may be added to a sample. Blood may also be centrifuged to obtain serum for testing.

A method of differentiating an inflammatory disease from a non-inflammatory disease, comprising: (a) contacting a biological sample from a human suspected of having said disorder with an antibody or antigen-binding fragment thereof, described herein under conditions that allow binding of the antibody or antigen-binding fragment thereof, to hepcidin; and (b) detecting and/or quantitating the hepcidin bound to the antibody or antigen-binding fragment thereof, wherein the amount of hepcidin, as quantitated in (b), above a threshold level indicates the presence of inflammatory disease and below the threshold level indicates the absence of inflammatory disease. In some instances, the biological sample is treated/processed prior to use in the described kit assay. For example, EDTA or heparin may be added to a sample. Blood may also be centrifuged to obtain serum for testing.

A “subject” (e.g., a mammal such as a human or a non-human animal such as a primate, rodent, cow, horse, pig, sheep, etc.) according to one embodiment of the present application, is a mammal who exhibits one or more clinical manifestations and/or symptoms of a disease or disorder described herein.

Also provided herein are diagnostic methods utilizing an antibody described herein to detect a hepcidin-related disease or disorder, or an iron-related disease or disorder. In certain instances, the methods detect a disease or disorder associated with an elevated level of hepcidin, a reduced level of hepcidin, an elevated level of iron, a reduced level of iron, or a combination thereof. Hepcidin-related disorders, inflammatory diseases, and diseases or disorders of iron homeostasis for which the methods may be applied include but are not limited to African iron overload, alpha thalassemia, Alzheimer's disease, anemia, anemia of cancer, anemia of chronic disease, anemia of inflammation, arteriosclerosis or atherosclerosis (including coronary artery disease, cerebrovascular disease or peripheral occlusive arterial disease), ataxias, ataxias related to iron, atransferrinemia, cancer, ceruloplasmin deficiency, chemotherapy-induced anemia, chronic renal/kidney disease (stage I, II, III, IV or V), including end stage renal disease or chronic renal/kidney failure, acute kidney injury (AKI), cirrhosis of liver, classic hemochromatosis, collagen-induced arthritis (CIA), conditions with hepcidin excess (elevated hepcidin), congenital dyserythropoietic anemia, congestive heart failure, Crohn's disease, Celiac disease, inflammatory bowel disease (IBD), diabetes, disorders of iron biodistribution, disorders of iron homeostasis, disorders of iron metabolism, ferroportin disease, ferroportin mutation hemochromatosis, folate deficiency, Friedrich's ataxia, funicular myelosis, gracile syndrome, a H. pylori infection or other bacterial infection, Hallervordan Spatz disease, hemochromatosis, hemochromatosis resulting from mutations in transferrin receptor 2, hemoglobinopathies, hepatitis, hepatitis (Brock), hepatitis C, hepatocellular carcinoma, hepcidin deficiency, hereditary hemochromatosis, HIV or other viral illnesses, Huntington's disease, hyperferritinemia, hypochromic microcytic anemia, hypoferremia, insulin resistance, iron deficiency anemia, iron deficiency disorders, iron overload disorders, iron-deficiency conditions with hepcidin excess, juvenile hemochromatosis (HFE2), multiple sclerosis, mutation in transferrin receptor 2, HFE, hemojuvelin, ferroportin, TMPRSS6 (IRIDA), or other genes of iron metabolism, neonatal hemochromatosis, neurodegenerative diseases related to iron, osteopenia, osteoporosis pancreatitis, Pantothenate kinase-associated neurodegeneration, Parkinson's disease, pellagra, pica, porphyria, porphyria cutanea tarda, pseudoencephalitis, pulmonary hemosiderosis, red blood cell disorders, rheumatoid arthritis, sepsis, sideroblastic anemia, systemic lupus erythematosus, thalassemia, thalassemia intermedia, transfusional iron overload, tumors, vasculitis, vitamin B6 deficiency, vitamin B12 deficiency, and/or Wilson's disease.

SEQUENCES
Human hepcidin peptide (hepcidin-25, hep-25,
Hep-25, hHepcidin-25): (25aa)
(SEQ ID NO: 1)
DTHFPICIFCCGCCHRSKCGMCCKT..
Human hepcidin-20 peptide (hepcidin-20, hep-20,
Hep-25, hHepcidin-20): (20aa)
(SEQ ID NO: 2)
ICIFCCGCCHRSKCGMCCKT..
Human hepcidin 22 peptide (hepcidin-22, hep-22,
Hep-22, hHepcidin-22): (22aa)
(SEQ ID NO: 3)
FPICIFCCGCCHRSKCGMCCKT..
Variable Heavy and Light Chain Nucleic Acid and
Amino Acid Sequences of Hepcidin MAb 583.
583 VH nucleic acid sequence:
(SEQ ID NO: 4)
GCTTCTGGGTATACCTTCACAAACTATGGAATGAACGGCTGGATAAACAC
CTACACTGGAGAGCCAACATATTTCTGTACAACGTACGCTACTAGCTGGT
ACTGGGGC.
583 VH amino acid sequence:
(SEQ ID NO: 5)
Ala Ser Gly Tyr Thr Phe Thr Asn Tyr Gly Met Asn
Gly Trp Ile Asn Thr Tyr Thr Gly Glu Pro Thr Tyr
Phe Cys Thr Thr Tyr Ala Thr Ser Trp Tyr Trp Gly.
583 VL nucleic acid sequence:
(SEQ ID NO: 6)
GCCAGTGAAAGTGTTGATAGTTATGGCAATAGTTTTATGCACATCTATCG
TGCATCCAACCTATACTGTCAGCAAAGTAATGAGGATCTGACGTTCGGT.
583 VL amino acid sequence:
(SEQ ID NO: 7)
Ala Ser Glu Ser Val Asp Ser Tyr Gly Asn Ser Phe
Met His Ile Tyr Arg Ala Ser Asn Leu Tyr Cys Gln
Gln Ser Asn Glu Asp Leu Thr Phe Gly.
CDR-1, CDR-2, and CDR-3 polynucleotide sequences
of Variable Heavy and Light Chains of
Hepcidin MAb 583.
Variable Heavy Chain CDRs
583 CDR-1:
(SEQ ID NO: 8)
GGGTATACCTTCACAAACTATGGA;
583 CDR-2:
(SEQ ID NO: 9)
ATAAACACCTACACTGGAGAGCCA;
and
583 CDR-3:
(SEQ ID NO: 10)
ACAACGTACGCTACTAGCTGGTAC.
Variable Light Chain CDRs
583 CDR-1:
(SEQ ID NO: 11)
GAAAGTGTTGATAGTTATGGCAATAGTTTT;
583 CDR-2:
(SEQ ID NO: 12)
CGTGCATCC;
and
583 CDR-3:
(SEQ ID NO: 13)
CAGCAAAGTAATGAGGATCTGACG.

### EXAMPLES

The application may be better understood by reference to the following non-limiting examples, which are provided as exemplary embodiments of the application. The following examples are presented in order to more fully illustrate embodiments and should in no way be construed, however, as limiting the broad scope of the application.

### Example 1: Intrinsic Hepcidin IDx™ ELISA

The Intrinsic LifeSciences (ILS) Hepcidin IDx™ ELISA kit is a competitive binding assay between Hepcidin-25 in the test specimen and a biologically active biotinylated human hepcidin-25 tracer for a constant number of high affinity anti-hepcidin-25 N-terminal-specific mAb binding sites. To begin, purified hepcidin-25 standard, hepcidin-25 controls (Control 1 and Control 2), and patient samples (all in duplicate) are added to the mAb coated microwell strip plate and incubated with biotinylated hepcidin-25 tracer for 60 minutes. The biotinylated hepcidin-25 tracer competes with native or reference hepcidin for a fixed number of N-terminal specific antibody binding sites. Thus, the amount of biotinylated hepcidin-25 tracer bound progressively decreases with increasing concentration of native serum hepcidin bound from the patient sample. Unbound biotinylated hepcidin-25 tracer is washed away and streptavidin-horseradish peroxidase (HRP) conjugate is added to the wells. The wells are incubated for 30 minutes and washed to remove unbound streptavidin-HRP. TMB substrate is added for 15 minutes and the reaction is stopped with the addition of stop solution. Absorbance at 450 nm is measured using a microwell plate reader and the data recorded.

| MATERIALS PROVIDED | | 96 Tests |
|---|---|---|
| 1. | Hepcidin-25 mAb coated wells, 96 Microwell Strip | 12 × 8 × 1 |
| | Plate | |
| 2. | Hepcidin-25 Standard, 8 vials ready to use | 8 × 0.5 mL |
| 3. | Hepcidin-25 Control 1, 1 vial ready to use | 0.5 mL |
| 4. | Hepcidin-25 Control 2, 1 vial ready to use | 0.5 mL |
| 5. | Biotinylated Hepcidin-25 Tracer, 1 bottle ready to use | 12 mL |
| 6. | Streptavidin-HRP Conjugate, 1 bottle ready to use | 12 mL |
| 7. | TMB Substrate, 1 bottle ready to use | 12 mL |
| 8. | Stop Solution, 1 bottle ready to use | 12 mL |
| 9. | Wash Buffer, 1 bottle of 20X solution, dilute before use | 25 mL |
| 10. | Sample Diluent, 1 bottle ready to use | 3 mL |
| 11. | Polypropylene (PP) 96 Microwell Plate, 2 adhesive | 1 package |
| | covers | |

Materials Not Provided

Distilled or deionized water

Precision pipettes (single and multichannel), disposable pipette tips (100 μl and 1000 μl)

Microwell plate reader capable of reading absorbance at 450 nm, benchtop centrifuge.

Flat-head vortex mixer, microplate shaker, absorbent paper.

Curve fitting software, graph paper.

Specimen Collection and Handling

Blood specimens may be collected in serum, serum separator, lithium or sodium heparin plasma or EDTA plasma tubes. Serum and plasma specimens may be stored refrigerated at 2-8° C. for up to 24 hours from the time of draw.

If storage is required for more than 24 hours, the plasma or serum may be stored frozen at −20° C. to −80° C. for up to one year. Avoid multiple freeze-thaw cycles.

Prior to assay, frozen sera or plasma should reach room temperature. If visible precipitates are present, gently mix and centrifuge the sample prior to use. Do not use hemolyzed, contaminated or lipemic samples.

Reagent Preparation

All reagents and specimens must be allowed to reach room temperature before use. All reagents must be gently mixed without foaming. Once the procedure has started, all steps should be completed without interruption. Prepare 1× Wash Buffer by adding the contents of the bottle (25 mL, 20λ) to 475 mL of distilled or deionized water. Store Wash Buffer at room temperature (18-24° C.).

Standard Assay Procedure

For best results, premix the samples, standards, controls and tracer for the ELISA in the 96-well polypropylene (PP) plate provided in the kit.

Using a multichannel pipette, transfer 110 μl of biotinylated hepcidin-25 tracer to required wells of PP plate.

Transfer 22 μl of each hepcidin standard, sample(s) and each hepcidin control to the PP plate. If a patient sample requires further dilution see the instructions for diluting samples described below.

Using a multichannel pipette, carefully mix the solution. Transfer 120 μl of this solution from the PP plate to the corresponding wells in the 96 microwell strip plate.

Incubate the microwell strip plate for 60 minutes at room temperature, with agitation (350 rpm). Briskly shake out the contents of the wells into a waste reservoir.

Dispense 300 μl of 1× Wash Buffer into each well, and then briskly shake out the Wash Buffer into a waste reservoir. Strike the wells sharply on absorbent paper to remove residual droplets. Repeat 2 additional times for a total of 3 washes.

Dispense 100 μl of Streptavidin-HRP Conjugate into each well.

Incubate 30 minutes at room temperature with agitation (350 rpm). Briskly shake out the contents of the wells into a waste reservoir.

Dispense 300 μl of 1× Wash Buffer into each well, and then briskly shake out the Wash Buffer into a waste reservoir. Strike the wells sharply on absorbent paper to remove residual droplets. Repeat 2 additional times for a total of 3 washes.

Using a multichannel pipette, dispense 100 μl of TMB Substrate into each well. Incubate for 15 minutes at room temperature, preferably in the dark.

Dispense 50 μl of Stop Solution into each well to stop the enzymatic reaction. Carefully mix plate contents for 20-30 seconds.

Read absorbance at 450 nm within 15 minutes of addition of the stop solution.

Instructions for Diluting Samples

If the hepcidin concentration in the sample approaches or exceeds the upper limit of quantitation (approximately 250 ng/ml), dilute the sample and re-run the ELISA (see WARNINGS AND PRECAUTIONS). Perform the dilutions in the Sample Diluent (supplied) in a low protein binding 96 microwell plate (not included) or in individual low protein binding microcentrifuge tubes (not included). See sample dilution example depicted in table below. Transfer 22 μl of the diluted sample(s) to the 96-well PP plate as described in STANDARD ASSAY PROCEDURE (above).

| Sample Dilution Factor | 2 | 5 | 10 |
|---|---|---|---|
| Volume Sample (μl) | 15.0 | 6.0 | 3.0 |
| Volume Sample Diluent (μl) | 15.0 | 24.0 | 27.0 |
| Total Sample Volume (μl) | 30.0 | 30.0 | 30.0 |

When performing the data calculations, always multiply the sample results by the sample dilution factor.

Optional Assay Procedure

Prior to performing assay, bring all reagents to room temperature. Gently mix all reagents before use.

Dispense 20 μl of Hepcidin standards, controls and samples into each well.

Dispense 100 μl of Biotinylated Hepcidin-25 Tracer, into each well.

Incubation #1—

Incubate microwell strip plate for 60 minutes at room temperature, with agitation (350 rpm). Briskly shake out the contents of the wells into a waste reservoir.

Wash #1—

Dispense 300 μl of 1× Wash Buffer into each well, and then briskly shake out the Wash Buffer into a waste reservoir. Strike the wells sharply on absorbent paper to remove residual droplets. Repeat 2 additional times for a total of 3 washes.

Dispense 100l of Streptavidin-horseradish peroxidase (HRP) Conjugate into each well.

Incubation #2—

Incubate 30 minutes at room temperature with agitation (350 rpm). Briskly shake out the contents of the wells into a waste reservoir.

Wash #2—

Dispense 300 μl of 1× Wash Buffer into each well, and then briskly shake out the Wash Buffer into a waste reservoir. Strike the wells sharply on absorbent paper to remove residual droplets. Repeat 2 additional times for a total of 3 washes.

Using a multichannel pipette, dispense 100 μl of TMB (3,3′,5,5′-Tetramethylbenzidine) Substrate into each well.

Incubation #3—

Incubate for 15 minutes at room temperature, preferably in the dark.

Stop—

Dispense 50l of Stop Solution into each well to stop the enzymatic reaction. Carefully mix plate contents for 20-30 seconds.

Read absorbance at 450 nm within 15 minutes of addition of the stop solution.

Calculation of Results

Software Calculation.

A standard curve is generated and sample hepcidin concentration determined using either Four Parameter logistic (4PL) or Point-to-Point curve fitting algorithms with GRAPHPAD PRISM® (Version 6.01) software (world wide web site: graphad.com). This test was developed and validated using 4PL curve fitting. Both methods yield very similar results. We recommend one method be used consistently once chosen.

Manual Calculation.

To construct the standard curve, plot the absorbance (OD 450 nm) for the Hepcidin-25 standards (vertical axis) versus Hepcidin-25 standard concentrations (horizontal axis) on a linear graph paper. Draw the best curve through the points. Read the absorbance for controls and each unknown sample from the curve and record.

The calculations can also be conducted using an appropriate computer program.

Example of Hepcidin Standard Curve Data

| Hepcidin-25 | Concentration | Absorbance |
|---|---|---|
| Standard | (ng/mL) | (450 nm) |
| 1 | 0 | 2.36 |
| 2 | 2.5 | 2.14 |
| 3 | 10 | 1.61 |
| 4 | 25 | 0.90 |
| 5 | 50 | 0.47 |
| 6 | 100 | 0.24 |
| 7 | 250 | 0.10 |
| 8 | 1000 | 0.03 |

Correlation of Serum Hepcidin with Plasma Hepcidin

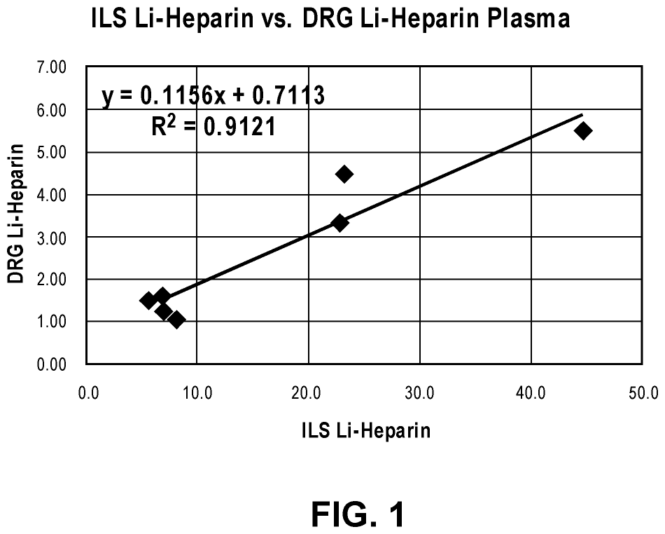

The graph of FIG. 1 represents regression analysis of serum versus plasma hepcidin from 8 human blood samples (low, medium, high hepcidin-25) obtained and prepared as serum, lithium heparin plasma, and EDTA plasma using standard procedures. Hepcidin-25 concentrations were measured using the Intrinsic Hepcidin IDx™ ELISA kit. A strong correlation was observed between serum and EDTA plasma (•, y=1.096x−1.25, R²=0.994) and serum and lithium heparin plasma (▪, y=0.946x+0.19, R²=0.994). See, FIG. 1.

| Sample ID | Serum | Plasma EDTA | Plasma-Li-Heparin |
|---|---|---|---|
| Patient 1 | 8.4 | 7.7 | 8.1 |
| Patient 2 | 7.0 | 7.4 | 7.1 |
| Patient 3 | 46.0 | 50.6 | 44.7 |
| Patient 4 | 6.9 | 5.0 | 5.9 |
| Patient 5 | 23.6 | 24.2 | 22.9 |
| Patient 6 | 26.5 | 25.7 | 23.1 |
| Patient 7 | 5.8 | 6.3 | 7.1 |
| Patient 8 | 5.8 | 5.8 | 5.7 |

Storage and Stability

Store the kit at 2-8° C.

Keep all microwell strips sealed in a dry bag with desiccant.

The reagents are stable until expiration date of the kit.

Do not expose test reagents to heat, sun, or strong light.

Warnings and Precautions

Potential biohazardous materials: The standards and controls contain human source components which have been tested and found non-reactive for hepatitis B surface antigen as well as HIV antibody with FDA licensed reagents. However, no test method can offer complete assurance that HIV, Hepatitis B virus or other infectious agents are absent. These reagents should be handled as Biosafety Level 2 material.

Do not pipette by mouth. Do not smoke, eat, or drink in the areas in which specimens or kit reagents are handled.

The components in this kit are an integral unit. Reagents from different lots should not be mixed. It is recommended that standards, control and patient samples be run in duplicate. Optimal results are obtained by strict adherence to this protocol.

Accurate and precise pipetting, careful washing and droplet removal, and exact timing at specified temperatures are essential. Deviation from recommendations may yield invalid data.

Check the Hepcidin-25 standard concentration value on each vial. This value might vary from lot-to-lot.

Patient samples with an Absorbance (A) 450 nm equal to or less than the A450 nm for the 250 ng/ml Hepcidin-25 standard (standard 7) should be diluted and re-run (See, Instructions For Diluting Samples).

### Example 2: Assay Coefficient of Variation (CV) and Hepcidin Gender Variances

Precision and accuracy are among the most important functional characteristics of any assay. Accuracy is difficult to assess if the assay suffers from poor reproducibility. The ability to reproduce a sample measurement from one well to another in a single assay and a sample measurement from one assay to the next is the first step to understanding the viability of the assay.

Commercially available immunoassays for hepcidin generally have an intra- and inter-assay coefficient of variation (CV) below 20%. It was an object of the present inventors to design a kit that has a more precise CV for greater accuracy of a hepcidin detection assay. More precise measurements allow for diagnosis of conditions associated with elevated or reduced levels of hepcidin with greater accuracy.

Serum samples were selected that contain low, medium, or high levels of endogenous hepcidin. These samples were run (n=16) in 3 independent assays. A CV was calculated for each sample in each assay. Intra-assay precision was calculated as the average CV across the assays and the inter-assay precision was calculated as the CV for all measurements (n=48). The average low, medium, and high sample was 22.2 ng/ml, 92.8 ng/ml, and 222.5 ng/ml, respectively. All intra- and inter-assay CVs identified by the present methods fall under 5%, which is very precise. Serum samples containing low, medium, or high levels of endogenous hepcidin were measured (n=16) in 3 assays. The average hepcidin value and associated coefficient of variation is listed in the following table.

| | Low | Medium | High |
|---|---|---|---|
| Hepcidin (ng/ml) | 22.2 | 92.8 | 222.5 |
| Intra CV* | 3.1% | 2.6% | 3.4% |
| Inter CV\hat{ } | 3.3% | 2.6% | 3.8% |

*Average n = 16; CV over 3 assays.
\hat{ }n = 48 over 3 assays.

The confirmation of excellent precision then allows the assessment of accuracy. Spike recovery is the most direct way of assessing accuracy due to the fact that the spiked quantities are known and no ‘gold-standard’ comparison assay is required. Two matrices were selected for spiking: assay buffer and serum. The assay buffer represents a simple matrix in the absence of interfering substances. The serum sample selected represents a complex, clinically relevant matrix devoid of endogenous hepcidin. Acceptance criteria for spike recovery dictate a relative error (RE)±20% for each level tested. Hepcidin was spiked into each matrix at levels across the analytical range.

Spike Recovery.

The two matrices selected for spike recovery were assay buffer and a serum sample containing no endogenous hepcidin. Hepcidin was spiked into these matrices across the analytical range. Relative errors were calculated for each measurement. The average relative error (RE) was −12% and −4% for buff and serum, respectively as shown in the following table.

| Hepcidin | Sample Matrix | |
|---|---|---|
| (ng/ml) | Buffer | Serum |
| 5 | 10% | 12% |
| 10 | −15% | 0% |
| 25 | −16% | −5% |
| 100 | −19% | −21% |
| 250 | −19% | −5% |
| AVE | −12% | −4% |

The average RE was −12% and −4% for buffer and serum, respectively.

Healthy, first time blood donor data can be taken to establish a reference range. Since male and female hepcidin values are significantly different from each other, the reference ranges are calculated separately. The mean hepcidin for females (n=148) and males (n=142) was determined to be 21.9 ng/ml and 41.5 ng/ml, respectively. The 5% to 95% quantiles ranged from 2.6 to 59.5 ng/ml and 7.3 to 104.3 ng/ml for females and males, respectively, as seen in the following table which provides data for the mean, median and select quantiles.

| Reference | N | Mean | Median | 5% | 50% | 95% |
|---|---|---|---|---|---|---|
| All | 290 | 31.5 | 23.4 | 3.1 | 23.4 | 91.4 |
| Female | 148 | 21.9 | 14.5 | 2.6 | 14.5 | 59.5 |
| Male | 142 | 41.5 | 32.4 | 7.3 | 32.4 | 104.3 |

Another important characteristic of a robust assay is dilutional linearity. This involves the measurement of high hepcidin serum samples serially diluted in assay buffer. Acceptable linearity occurs when the values for the diluted samples compared to the undiluted sample measurements yield relative errors ±20%. Two high hepcidin serum samples were diluted 1:2 and 1:4 in assay buffer and measured alongside undiluted sample. The average relative errors for the 1:2 and 1:4 dilutions were −2% and −1%, respectively, as shown in the following table.

| | Dilution: | | | | |
|---|---|---|---|---|---|
| | Neat | 1:2 | | 1:4 | |
| | Measured | Measured | | Measured | |
| Sample ID | (ng/ml) | (ng/ml) | RE | (ng/ml) | RE |
| Serum 1 | 363.6 | 175.0 | −4% | 84.1 | −8% |
| Serum 2 | 295.5 | 146.4 | −1% | 77.6 | 5% |
| | | AVE | −2% | AVE | −1% |

Dilutional Linearity: two serum samples were diluted 1:2 and 1:4; then, hepcidin was measured using the ILS Hepcidin IDx™ ELISA kit, the current kit described in this patent. The relative errors were calculated form comparison to an undiluted measurement. The commercially available test gave relative errors of 3% and −3% for 1:2 and 1:4 dilution, respectively.

The experiments and data presented above show clearly that the ILS Hepcidin IDx™ ELISA kit has excellent technical characteristics and utility for clinical analysis of patient samples for hepcidin-25 and following clinical studies, for diagnosis of genetic and acquired anemias and iron overloading diseases.

### Example 3: Comparison of Current Kit with DRG® Commercial Kit

The current kit described in the present application was compared to the DRG® Hepcidin 25 bioactive ELISA (EIA-5258), version 4.1, revised Apr. 28, 2014, DRG International, Inc., USA, using the manufacturer's instructions. Briefly, the materials and protocol for the DRG® kit are as follows:

DRG® Reagents Provided with the Kit

Microtiterwells, 12×8 (break apart) strips, 96 wells; Wells coated with anti-Hepcidin-25 antibody (monoclonal).

Standard (Standard 0-5), 6 vials (lyophilized), 0.2 mL; Concentrations: 0-2-6.5-25-45-80 ng/mL; Conversion: 1 ng/mL=0.358 nmol/L Contains non-mercury preservative.

Control Low & High, 2 vials, (lyophilized), 0.2 mL, For control values and ranges, refer to vial label or QC-Datasheet.

Assay Buffer, 1 vial, 14 mL, ready to use, Contains non-mercury preservative.

Enzyme Conjugate, 1 vial, 7 mL, ready to use, Hepcidin-25 conjugated to biotin; Contains non-mercury preservative.

Enzyme Complex, 1 vial, 14 mL, ready to use, Streptavidin conjugated to HRP; Contains non-mercury preservative.

Substrate Solution, 1 vial, 14 mL, ready to use, Tetramethylbenzidine (TMB).

Stop Solution, 1 vial, 14 mL, ready to use, contains 0.5 M H2SO4.

Wash Solution, 1 vial, 30 mL (40× concentrated).

Bring all reagents and required number of strips to room temperature prior to use.

DRG® Standards Provided with the Kit

Reconstitute the lyophilized contents of the standard vial with 0.2 mL deionized water and let stand for 10 minutes in minimum. Mix the standard several times before use.

DRG® Controls

Reconstitute the lyophilized content with 0.2 mL deionized water and let stand for 10 minutes in minimum. Mix the controls several times before use.

DRG® Wash Solution

Add deionized water to the 40× concentrated Wash Solution. Dilute 30 mL of concentrated Wash Solution with 1170 mL deionized water to a final volume of 1200 mL.

DRG® Specimen Collection and Preparation

Serum or heparin plasma can be used in this assay. EDTA- and citrate plasma results in decreased (˜20%) values.

Specimen Collection

Serum: Collect blood by venipuncture (e.g., Sarstedt Monovette for serum), allow to clot, and separate serum by centrifugation at room temperature. Do not centrifuge before complete clotting has occurred. Samples containing anticoagulant may require increased clotting time.

Plasma: Whole blood should be collected into centrifuge tubes containing anti-coagulant (e.g., Sarstedt Monovette with the appropriate plasma preparation) and centrifuged immediately after collection.

Specimen Dilution

If in an initial assay, a specimen is found to contain more than the highest standard, the specimens can be diluted with Assay Buffer and reassayed as described in Assay Procedure. For the calculation of the concentrations this dilution factor has to be taken into account.

Example:

a) dilution 1:10: 10 μL Serum+90 μL Assay Buffer (mix thoroughly)
b) dilution 1:100: 10 μL dilution a) 1:10+90 μL Assay Buffer (mix thoroughly).

Assay Procedure

Each run must include a standard curve.

1. Secure the desired number of Microtiter wells in the frame holder.

2. Dispense 100 μL of Assay Buffer into appropriate wells.

3. Dispense 20 μL of each Standard, Controls and samples with new disposable tips into appropriate wells.

4. Dispense 50 μL of Enzyme Conjugate into each well. Thoroughly mix for 10 seconds. It is important to have a complete mixing in this step.

5. Incubate for 60 minutes at room temperature under agitation (300-700 rpm).

6. Briskly shake out the contents of the wells. Rinse the wells 3 times with 400 μL diluted Wash Solution per well (if a plate washer is used) or 4 times with 300 μL diluted Wash Solution per well for manual washing. Strike the wells sharply on absorbent paper to remove residual droplets.

7. Dispense 100 μL of Enzyme Complex into appropriate wells.

8. Incubate for 30 minutes at room temperature without agitation.

9. Briskly shake out the contents of the wells. Rinse the wells 3 times with 400 μL diluted Wash Solution per well (if a plate washer is used) or 4 times with 300 μL diluted Wash Solution per well for manual washing. Strike the wells sharply on absorbent paper to remove residual droplets.

10. Add 100 μL of Substrate Solution to each well.

11. Incubate for 20 minutes at room temperature.

12. Stop the enzymatic reaction by adding 100 μL of Stop Solution to each well.

13. Determine the absorbance (OD) of each well at 450±10 nm with a microtiter plate reader. It is recommended that the wells be read within 10 minutes after adding the Stop

Solution.

Calculation of Results

Calculate the average absorbance values for each set of standards, controls and samples. Using semi-logarithmic graph paper, construct a standard curve by plotting the mean absorbance obtained from each standard against its concentration with absorbance value on the vertical (Y) axis and concentration on the horizontal (X) axis.

Using the mean absorbance value for each sample determine the corresponding concentration from the standard curve.

Automated method: The results in the IFU have been calculated automatically using a 4 PL (4 Parameter Logistics) curve fit. 4 Parameter Logistics is the preferred method. Other data reduction functions may give slightly different results. The concentration of the samples can be read directly from this standard curve. Samples with concentrations higher than that of the highest standard have to be further diluted or reported as >80 ng/mL. Comparison of Results between the Current Kit and the DRG® hepcidin-25 bioactive kit.

FIG. 1 shows a linear regression of the plasma hepcidin results from the ILS Hepcidin IDx™ ELISA kit and the DRG® comparator kit. The slope of the regression shows that the DRG® kit measures an approximately 9-fold lower hepcidin concentration in HIPPA compliant Li-heparin samples compared to the ILS Hepcidin IDx™ ELISA kit. The correlation was good between the two kits with an R²=0.91. The discrepancy in plasma hepcidin concentrations led us to measure the reference standards from present kit using the DRG® comparator kit and the reference standards from the DRG® kit with the ILS Hepcidin IDx™ ELISA.

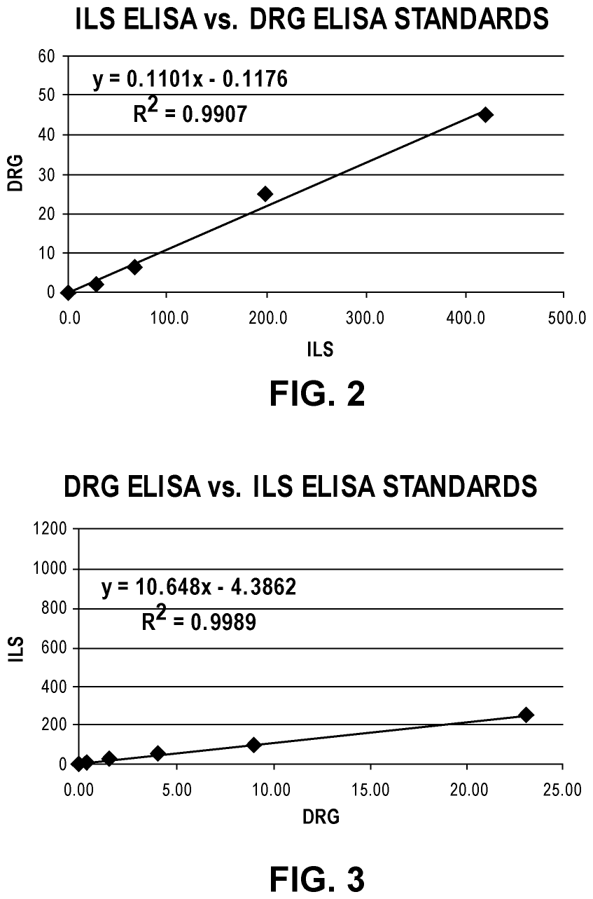

FIG. 2 shows the measurement of the DRG® comparator reference standards with the standards of the present kit. The results of the comparison of the standards are shown in FIG. 2 as a linear regression between the present kit and the DRG® comparator kit. The regression is describe by the equation y=0.1101x−0.1176, R²=0.9907. While the results are highly correlated between the present kit and the DRG kit, they clearly show that the DRG® standards are approximately 9-fold lower than those from the present invention for the same described concentrations of standards.

FIG. 3 compares the reference standards from the present kit measured using the DRG® comparator kit. The results of this comparison is shown in FIG. 3 as a linear regression described by the equation y=10.648x−4.3862, R²=0.9989. This comparison confirms that the DRG® reference standards are 10.6-fold lower than labelled on the reference standards stated in the DRG® kit. The standards used in the present kit contain the true concentrations of hepcidin as labelled indicating that the DRG® kit uses reference standards that have be adjusted to yield similar results to a MALDI-TOF Mass Spec assay.

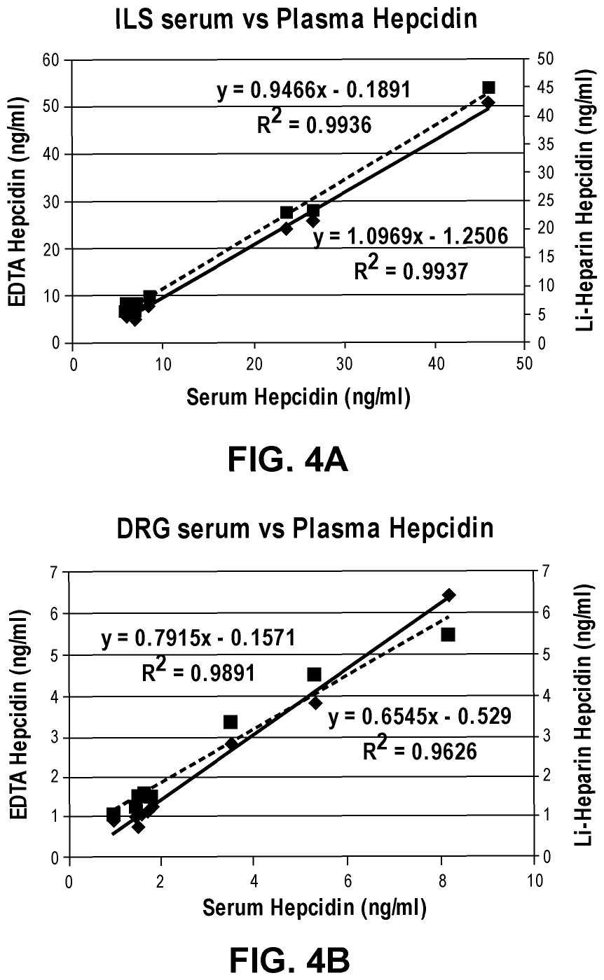

FIGS. 4A and 4B demonstrate that the present kit is an improvement over the existing commercial DRG® kit. These figures show the relative effects of the sample matrix on the respective hepcidin ELISAs. FIG. 4A shows the results from the present kit from the same blood sample prepared as serum versus the same sample prepared as plasma using a Li-heparin blood tube or using K₂-EDTA, respectively. Inspection of the linear regression equations show that samples prepared as plasma using either Li-heparin or K2-EDTA yields very similar slopes in both plasma matrices compared to serum that is approximately ±10% the slope of a serum sample.

FIG. 4B shows the same samples prepared as serum or Li-heparin or K₂-EDTA. The regression analysis and equations for both Li-heparin and K₂-EDTA show much greater effects (±34%) of the sample matrix type relative to serum compared to the present kit (Li-heparin; y=0.7915x−0.1571; K₂-EDTA; y=0.6545x−0.529). The greater agreement between plasma and serum hepcidin concentrations in the present kit is a clear improvement over the DRG® comparator kit.

In conclusion, the present kit of this invention represents a significant technical improvement over the comparator DRG® Hepcidin-25 (Bioactive) ELISA kit (EIA-5258) and the data contained in DRG®'s newer version (EIA-5782).

The present kit utilizes reference standards that are formulated at the concentrations shown on the reference standard tubes in the present kit and are not adjusted artificially so that results mimic those of a MALDI-TOF MS test that may suffer from significant pre-analytical error itself (FIGS. 2 and 3). Indeed, one measuring hepcidin levels in samples using the DRG® Hepcidin-25 (Bioactive) ELISA kit (EIA-5258) and DRG®'s newer version (EIA-5782) would obtain incorrect results because of the standards provided in those kits.

In addition, the present kit yields consistent results regardless of the anti-coagulant used to prepare the plasma as compared to the DRG® comparator kit (FIGS. 4A and 4B).

The present kit also has a 3-fold larger analytical measurement range of zero to 250 ng/ml versus 80 ng/ml for the DRG® comparator kit we used in the comparison as well as the newer version of the DRG® comparator kit. This allows measurement of a greater range of samples from diseases known to increase plasma hepcidin concentrations such as chronic kidney disease (CKD), dialysis patient samples, and other inflammatory diseases.

The present kit has better intra- and inter-assay coefficients of variation (CV) that the DRG® comparator kit.

The reference ranges reported for the present kit show a clear gender difference between females and males that is not observed using the DRG® kit. Gender differences are an expected feature of hepcidin reference ranges for any excellent validated hepcidin assay.

A key distinction between the Intrinsic Hepcidin IDx™ ELISA kit and the DRG® comparator kit is that the present kits are manufactured in an FDA-approved cGMP facility under FDA compliant Design Controls and Quality Systems. The present kits are suitable for FDA clearance following multi-center clinical studies.

A key advantage of the present invention is the unique, high-affinity monoclonal antibody, mAb583 used to allow sensitive and specific binding of the N-terminus of bioactive Hepcidin-25 and other isoforms with lower bioactivity against ferroportin, hepcidin's receptor and main iron transporter in humans. Mab583 has excellent affinity to hepcidin-25 with a Biacore binding constant estimated at 7-8 pM. This mAb583 antibody is stored as an immortal hybridoma that is stable and yields commercial quantities of the mAb using standard techniques and bioreactor technologies.

In contrast little is known about the antibody used in the comparator kit other than it than that it binds the C-terminus of hepcidin. The C-terminus and in fact di-sulfide pairing pattern in the C-terminus has been shown to have little effect on bioactivity and thus, the test relies on an binding epitope that is not specific for bioactive forms of hepcidin. The mAb583 antibody used as the key reagent in the present kit is a major improvement and superior considering hepcidin-25 and the key role of the N-terminus. The antibody used in the DRG® comparator kit is less reliable in terms of the known effect of structure and function of hepcidin-25.

Taken together these Examples clearly show that the present kit is an improvement over existing commercially available kits.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A kit comprising
a) a 96-microwell strip plate pre-coated with an anti-hepcidin antibody;
b) a hepcidin-25 standard;
c) a first hepcidin-25 control;
d) a second hepcidin-25 control;
e) a biotinylated hepcidin-25 tracer;
f) a streptavidin horseradish peroxidase conjugate;
g) a 3,3′,5,5′-Tetramethylbenzidine (TMB) substrate;
h) a stop solution;
i) a wash buffer; and
j) a sample diluent.

2. The kit of claim 1, wherein the microwell strip plate is a polystyrene microwell strip plate.

3. The kit of claim 1, further comprising two adhesive covers for the microwell strip plate.

4. The kit of claim 1, wherein the antibody of a) is an antibody having a variable heavy chain set forth as SEQ ID NO: 5 and a variable light chain set forth as SEQ ID NO: 7.

5. The kit of claim 1, comprising 8 vials of hepcidin-25 standard.

6. The kit of claim 5, wherein each vial comprises 0.5 mL of hepcidin-25 standard.

7. The kit of claim 1, comprising 1 vial of the first hepcidin-25 control.

8. The kit of claim 7, wherein the vial of the first hepcidin-25 control comprises 0.5 mL of reagent.

9. The kit of claim 1, comprising 1 vial of the second hepcidin-25 control.

10. The kit of claim 9, wherein the vial of the second hepcidin-25 control comprises 0.5 mL of reagent.

11. The kit of claim 1, comprising 1 bottle of biotinylated hepcidin-25 tracer.

12. The kit of claim 11, wherein the vial comprises 12 mL of biotinylated hepcidin-25 tracer.

13. The kit of claim 1, wherein the biotinylated hepcidin-25 tracer consists of: (i) a hepcidin peptide that is oxidatively folded; (ii) a hydrophilic spacer consisting of two (2-(2-Amino-Ethoxy) Ethoxy) Acetic Acid (AEEAc) residues, wherein the peptide of (i) is covalently linked to the hydrophilic spacer at the amino terminus of the peptide; and (iii) biotin covalently linked to the hydrophilic spacer of (ii).

14. The kit of claim 13, wherein the biotinylated hepcidin-25 tracer comprises a hepcidin peptide having an amino acid sequence set forth as SEQ ID NO: 1.

15. The kit of claim 1, comprising 1 bottle of Streptavidin-HRP Conjugate.

16. The kit of claim 15, wherein the bottle comprises 12 mL of Streptavidin-HRP Conjugate.

17. The kit of claim 1, comprising 1 bottle of Streptavidin-HRP Conjugate.

18. The kit of claim 17, wherein the bottle comprises 12 mL of Streptavidin-HRP Conjugate.

19. The kit of claim 1, comprising 1 bottle of TMB Substrate.

20. The kit of claim 19, wherein the bottle comprises 12 mL of TMB Substrate.

21. The kit of claim 1, comprising 1 bottle of Stop Solution.

22. The kit of claim 21, wherein the bottle comprises 12 mL of Stop Solution.

23. The kit of claim 1, comprising 1 bottle of Wash Buffer.

24. The kit of claim 23, wherein the bottle comprises 25 mL of Wash Buffer.

25. The kit of claim 23, comprising a 20× solution of the Wash Buffer.

26. The kit of claim 25, wherein the wash solution is diluted prior to use in the kit.

27. The kit of claim 1, comprising 1 bottle of Sample Diluent.

28. The kit of claim 27, wherein the bottle comprises 3 mL of Sample Diluent.

29. The kit of any one of claims 1-28, further comprising instructions for use.

30. The kit of claim 29, wherein the instructions comprise identification of a biological sample for use in the kit.

31. The kit of claim 30, further comprising one or more collection means for collection of the biological sample.

32. The kit of claim 31, wherein the one or more collection means comprises a syringe, a needle, a cup, a swab or a combination thereof.

33. The kit of claim 30, wherein the biological sample comprises a blood sample, a tissue sample or a urine sample.

34. The kit of any one of claims 1-33, further comprising a label.

35. The kit of claim 34, for use in detecting one or more diseases or disorders associated with an elevated level of hepcidin, a reduced level of hepcidin, an elevated level of iron, a reduced level of iron, or a combination thereof.

36. The kit of claim 35, wherein the one or more diseases or disorders associated with elevated level of hepcidin, a reduced level of hepcidin, an elevated level of iron, a reduced level of iron, or a combination thereof, is African iron overload, alpha thalassemia, Alzheimer's disease, anemia, anemia of cancer, anemia of chronic disease, anemia of inflammation, arteriosclerosis or atherosclerosis, ataxias, ataxias related to iron, atransferrinemia, cancer, ceruloplasmin deficiency, chemotherapy-induced anemia, chronic renal disease, including end stage renal disease or chronic renal/kidney failure, acute kidney injury (AKI), cirrhosis of liver, classic hemochromatosis, collagen-induced arthritis (CIA), congenital dyserythropoietic anemia, congestive heart failure, Crohn's disease, Celiac disease, inflammatory bowel disease (IBD), diabetes, a disorder of iron biodistribution, a disorder of iron homeostasis, a disorder of iron metabolism, ferroportin disease, ferroportin mutation hemochromatosis, folate deficiency, Friedrich's ataxia, funicular myelosis, gracile syndrome, a bacterial infection, Hallervordan Spatz disease, hemochromatosis, hemochromatosis resulting from mutations in transferrin receptor 2, hemoglobinopathies, hepatitis, hepatocellular carcinoma, hereditary hemochromatosis, a viral infection, Huntington's disease, hyperferritinemia, hypochromic microcytic anemia, hypoferremia, insulin resistance, iron deficiency anemia, an iron deficiency disorder, an iron overload disorder, an iron-deficiency condition with hepcidin excess, juvenile hemochromatosis (HFE2), multiple sclerosis, mutation in transferrin receptor 2, HFE, hemojuvelin, ferroportin, TMPRSS6 (IRIDA), or other genes of iron metabolism, neonatal hemochromatosis, neurodegenerative diseases related to iron, osteopenia, osteoporosis pancreatitis, Pantothenate kinase-associated neurodegeneration, Parkinson's disease, pellagra, pica, porphyria, porphyria cutanea tarda, pseudoencephalitis, pulmonary hemosiderosis, a red blood cell disorder, rheumatoid arthritis, sepsis, sideroblastic anemia, systemic lupus erythematosus, thalassemia, thalassemia intermedia, transfusional iron overload, a tumor, vasculitis, vitamin B6 deficiency, vitamin B12 deficiency, Wilson's disease or a combination thereof.


SEQUENCE LISTING (13 sequences)

SEQ ID NO: 1 - protein, 25 aa, Homo sapiens
DTHFPICIFCCGCCHRSKCGMCCKT

SEQ ID NO: 2 - protein, 20 aa, Homo sapiens
ICIFCCGCCHRSKCGMCCKT

SEQ ID NO: 3 - protein, 22 aa, Homo sapiens
FPICIFCCGCCHRSKCGMCCKT

SEQ ID NO: 4 - DNA, 108 bp, Artificial Sequence
  Description of Artificial Sequence Synthetic polynucleotide
gcttctgggtataccttcacaaactatggaatgaacggctggataaacacctacactggagagccaacatatttctgtacaacgtacgctactagctggtactggggc

SEQ ID NO: 5 - protein, 36 aa, Artificial Sequence
  Description of Artificial Sequence Synthetic polypeptide
ASGYTFTNYGMNGWINTYTGEPTYFCTTYATSWYWG

SEQ ID NO: 6 - DNA, 99 bp, Artificial Sequence
  Description of Artificial Sequence Synthetic oligonucleotide
gccagtgaaagtgttgatagttatggcaatagttttatgcacatctatcgtgcatccaacctatactgtcagcaaagtaatgaggatctgacgttcggt

SEQ ID NO: 7 - protein, 33 aa, Artificial Sequence
  Description of Artificial Sequence Synthetic polypeptide
ASESVDSYGNSFMHIYRASNLYCQQSNEDLTFG

SEQ ID NO: 8 - DNA, 24 bp, Artificial Sequence
  Description of Artificial Sequence Synthetic oligonucleotide
gggtataccttcacaaactatgga

SEQ ID NO: 9 - DNA, 24 bp, Artificial Sequence
  Description of Artificial Sequence Synthetic oligonucleotide
ataaacacctacactggagagcca

SEQ ID NO: 10 - DNA, 24 bp, Artificial Sequence
  Description of Artificial Sequence Synthetic oligonucleotide
acaacgtacgctactagctggtac

SEQ ID NO: 11 - DNA, 30 bp, Artificial Sequence
  Description of Artificial Sequence Synthetic oligonucleotide
gaaagtgttgatagttatggcaatagtttt

SEQ ID NO: 12 - DNA, 9 bp, Artificial Sequence
  Description of Artificial Sequence Synthetic oligonucleotide
cgtgcatcc

SEQ ID NO: 13 - DNA, 24 bp, Artificial Sequence
  Description of Artificial Sequence Synthetic oligonucleotide
cagcaaagtaatgaggatctgacg